(19) United States
(12) Patent Application Publication
(10) Pub. No.: US 2018/0169123 A1
(43) Pub. Date: Jun. 21, 2018

(54) COMBINATION THERAPY WITH A FLAVAGLINE AND 2-DEOXYGLUCOSE

(71) Applicants: Ruprecht-Karls-Universitat Heidelberg, Heidelberg (DE); Deutsches Krebsforschungszentrum, Heidelberg (DE)

(72) Inventors: Magnus VON KNEBEL DOEBERITZ, Heidelberg (DE); Matthias KLOOR, Ludwigshafen (DE); Aysel AHADOVA, Heidelberg (DE); Juergen KOPITZ, Wiesloch (DE); Peter KRAMMER, Heidelberg (DE); Min LI-WEBER, Heidelberg (DE)

(21) Appl. No.: 15/738,378

(22) PCT Filed: Jun. 24, 2016
(86) PCT No.: PCT/EP2016/064691

§ 371 Date: Dec. 20, 2017

(30) Foreign Application Priority Data
Jun. 26, 2015 (EP) 15174087.5

(51) Int. Cl. A61K 31/7004; A61K 31/343; A61K 31/36; A61K 45/06; A61P 35/00

(52) U.S. Cl. CPC A61K 31/7004; A61K 31/343; A61K 2300/00; A61K 45/06; A61P 35/00; A61K 31/36

(57) ABSTRACT

The present invention relates to a combined preparation comprising 2-deoxyglucose and a flavagline and to the use of said combined preparation as a medicament or in the treatment of cancer. Moreover, the present invention relates to a flavagline for use in a combination therapy against cancer comprising administration of 2-deoxyglucose; and to 2-deoxyglucose for use in a combination therapy against cancer comprising administration of a flavagline. Also, the present invention relates to methods for treating cancer and to processes for the preparation of a combined preparation according to the invention.

The present invention relates to a combined preparation comprising 2-deoxyglucose and a flavagline and to the use of said combined preparation as a medicament and/or in the treatment of cancer. Moreover, the present invention relates to a flavagline for use in a combination therapy against cancer comprising administration of 2-deoxyglucose; and to 2-deoxyglucose for use in a combination therapy against cancer comprising administration of a flavagline. Also, the present invention relates to methods for treating cancer and to processes for the preparation of a combined preparation according to the invention.

Cancer constitutes the fourth leading cause of death in Western countries. As the average age in the Western population steadily rises, so do cancer-related deaths indicating that cancer will be one of the most common causes of death in the 21st century. The aggressive cancer cell phenotype is the result of a variety of genetic and epigenetic alterations leading to deregulation of intracellular signaling pathways. Cancer cells commonly fail to undergo so-called “programmed cell death” or “apoptosis”, a signaling process that plays a key role in preventing cell tissues from abnormal growth.

Three modes of cancer therapy are generally available. Curative surgery attempts to remove the tumor completely. This is only possible as long as there are no metastases. Sometimes surgery may be an option for the treatment of metastases if there are only few and they are easily accessible. Radiotherapy uses ionizing radiation, typically γ-radiation, to destroy the tumor. Radiation therapy is based on the principle that tumor cells with their high metabolic rates are especially susceptible to radiation induced cell damage. The anti-tumor effect of radiation therapy has to be weighed against the damage to the surrounding healthy tissue. Thus, possible tissue damage can rule out this option in some cases due to the damage to healthy tissues to be feared. Furthermore, radiation therapy is limited to cases where the primary tumor has not yet spread or where only few metastases are present.

The most commonly used—and in many instances the only available—systemic treatment for cancer is chemotherapy. For patients suffering from leukemia or from metastases of solid tumors, thus, chemotherapy is the only treatment option. Chemotherapeutic agents are cytotoxic for all rapidly dividing cells. As cancer cells usually divide more rapidly than other cells in the body, they are preferably killed by these agents. Common groups of chemotherapeutic agents are substances that inhibit cell division by interfering with the formation of the mitotic spindle or agents which damage the DNA, e.g. by alkylating the bases. Because all rapidly dividing cells are targeted by chemotherapeutic agents, their side effects are usually severe. Depending on the substance used, they include organ toxicity (e.g. heart or kidney), immunosuppression, neurotoxicity and anemia. Some groups of chemotherapeutic agents, e.g. alkylating agents, even have the potential to cause cancer. Due to these side effects, dosages have sometimes to be reduced or chemotherapy has to be discontinued completely. Furthermore, the side effects of chemotherapy often prohibit the treatment of patients in a bad general condition. Adding to all these problems is the often limited efficacy of chemotherapy. In some cases chemotherapy fails from the very beginning. In other cases, tumor cells become resistant during the course of treatment. To combat the emergence of resistant tumor cells and to limit the side effects of chemotherapy, combinations of different compounds with different modes of action are used. Nevertheless, the success of chemotherapy has been limited, especially in the treatment of solid tumors.

Recently, drugs have become available whose mode of action is not based on toxicity against rapidly dividing cells. These compounds show a higher specificity for cancer cells and thus less side effects than conventional chemotherapeutic agents. Imatinib is used for the specific treatment of chronic myelogenous leukemia. This compound specifically inhibits an abnormal tyrosine kinase which is the product of a fusion gene of bcr and abl. Because this kinase does not occur in non-malignant cells, treatment with Imatinib has only mild side effects. However, Imatinib is not used for the treatment of hematological cancers other than myelogenous leukemia. Rituximab is a monoclonal antibody directed against the cluster of differentiation 20 (CD20), which is widely expressed on B-cells. It is used for the treatment of B cell lymphomas in combination with conventional chemotherapy.

2-Deoxyglucose (2-DG) is a structural analog of glucose, in which the 2-OH group is replaced by a hydrogen atom. 2-DG is taken up by most cells via the glucose transporting systems and is phosphorylated by hexokinase to 2-deoxyglucose-6-phosphate, which, in turn inhibits phosphoglucoisomerase; as a consequence, glycolysis is inhibited. Cancer cells, in comparison with normal non-neoplastic cells, are particularly sensitive towards 2-DG, because they consume larger amounts of glucose for energy production. Since cancer cells, in particular those growing in areas of reduced oxygen tension in a solid tumor, are strongly dependent on glycolysis for energy production, 2-DG was proposed as a tumor therapeutic. Application of 2-DG in the treatment of human malignancies, however, is limited due to serious side effects caused by the high doses required for efficient therapy. These side effects comprise deregulation of blood glucose levels, progressive weight loss with lethargy, behavioral symptoms of hypoglycemia, restlessness, general neurological symptoms, excess vomiting, and cardiac side-effects (Singh et al., Strahlenther Onkol. 2005; 181(8):507-14; Marsh et al., Nutr Metab (Lond). 2008; 5:33.).

Flavaglines belong to the group of 1H-cyclopenta[b]benzofurans. Rocaglamide, a flavagline, and rocaglamide derivatives can be isolated from Aglaia Species. It has been demonstrated that they possess antiproliferative activity (see e.g. U.S. Pat. No. 4,539,414; Dhar et al., 1973 Indian J Exp Vol. 11, pages 43-54; King et al., 1982 J Chem Soc Chem Comm Vol. 20, pages 1150-1151; Lee et al., 1998 Chem Biol Interact Vol. 115, pages 215-228; Bohnenstengel et al., 1999, Z. Naturforsch [C]. Vol. 54, pages 55-60; Bohnenstaengel et al., 1999 Z Naturforsch [C] Vol 54, pages 1075-1083; Kim et al., 2006 Anticancer Agents Med Chem Vol. 6; pages 319-345). Rocaglamide derivatives have been shown to have an inhibitory effect on growth of a murine leukemia cell line (P-388), a human breast cancer cell line (BCI), a human myeloid leukemia cell line (M091), and a melanoma cell line with a BRAF(V600)-mutation in vitro and also in vivo (Hwang et al., 2014, J. Org. Chem. 69:3350-3358; Lee et al., 1998, Chem. Biol. Interact 115: 215-228; Santagata et al, 2013, Science 341:1238303; Boussemart et al., Nature. 2014, 513:105-109). Flavaglines were also shown to selectively target aneuploid cancer cells and non-transformed cells with cancer-associated genetic aberrations (Santagata et al, 2013, Science 341:1238303). Furthermore, flavaglines even may have cytoprotective activities on normal cells under certain circumstances, such as protection of selected non-malignant cells, as it has been reported that human peripheral blood T cells, B cells, NK cells, neutrophils, cardiomyocytes and murine hematopoietic stem and progenitor cells, may be less sensitive towards chemotherapy-induced cell death upon flavagline exposure. Flavaglines may under certain circumstances also alleviate inflammation- or drug-induced injury in neuronal tissue (Fahrig et al., Mol Pharmacol 2005, 67:1544-55; Bernard et al., PLoS One 2011, 6:e25302; Ribeiro et al., J Med Chem 2012, 55:10064-73; Becker et al., Cell Death Dis 2014; 5:e1000). Flavaglines have also been reported to under certain circumstances specifically block p53 expression induced by apoptosis-inducing chemotherapeutic drugs. Importantly, flavaglines do not appear to protect p53-deficient or -mutated cancer cells (Becker et al., Cell Death Dis 2014; 5:e1000).

In view of the above, there is a need in the art for an improved cancer therapy, in particular cancer therapy using the antimetabolite 2-deoxyglucose, which preferably avoids or largely avoids adverse effects.

The technical problem underlying the present invention can be regarded as the provision of means and methods for complying with the aforementioned needs. The said technical problem is solved by the embodiments characterized in the claims and herein below.

Accordingly, the present invention relates to a combined preparation comprising 2-deoxyglucose and a flavagline.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention. Moreover, if not otherwise noted, the term “about” relates to the indicated value±20%.

The term “combined preparation”, as referred to in this application, relates to a preparation comprising the pharmaceutically active compounds of the present invention in one preparation. Preferably, the combined preparation is comprised in a container, i.e. preferably, said container comprises all pharmaceutically active compounds of the present invention. Preferably, said container comprises the pharmaceutically active compounds of the present invention as separate formulations, i.e. preferably, one formulation of the 2-deoxyglucose and one formulation of the flavagline; more preferably, said container comprises the pharmaceutically active compounds of the present invention in a single formulation, e.g. preferably, as a two-layer-tablet, or the like. Most preferably, the combined preparation is a mixed formulation, i.e. preferably, the combined preparation comprises a mixture of the compounds of the present invention. As will be understood by the skilled person, the term “formulation” relates to a, preferably pharmaceutically acceptable, mixture of compounds, comprising or consisting of at least one pharmaceutically active compound of the present invention. Preferably, the combined preparation comprises a flavagline and 2-deoxyglucose in a single formulation, e.g. a tablet; more preferably, the combined preparation comprises a mixture of a flavagline and 2-deoxyglucose.

Preferably, the combined preparation is for separate or for combined administration. “Separate administration”, as used herein, relates to an administration wherein at least two of the pharmaceutically active compounds of the present invention are administered via different routes. E.g. one compound may be administered by enteral administration (e.g. orally), whereas a second compound is administered by parenteral administration (e.g. intravenously). Preferably, the combined preparation for separate administration comprises at least two physically separated preparations for separate administration, wherein each preparation contains at least one pharmaceutically active compound; said alternative is preferred e.g. in cases where the pharmaceutically active compounds of the combined preparation have to be administered by different routes, e.g. parenterally and orally, due to their chemical or physiological properties. Conversely, “combined administration” relates to an administration wherein the pharmaceutically active compounds of the present invention are administered via the same route, e.g. orally or intravenously.

Also preferably, the combined preparation is for simultaneous or for sequential administration. “Simultaneous administration”, as used herein, relates to an administration wherein the pharmaceutically active compounds of the present invention are administered at the same time, i.e., preferably, administration of the pharmaceutically active compounds starts within a time interval of less than 15 minutes, more preferably, within a time interval of less than 5 minutes. Most preferably, administration of the pharmaceutically active compounds starts at the same time, e.g. by swallowing a tablet comprising the pharmaceutically active compounds, or by applying an intravenous injection of a solution comprising the pharmaceutically active compounds. Conversely, “sequential administration”, as used herein, relates to an administration causing plasma concentrations of the pharmaceutically active compounds in a subject enabling the synergistic effect of the present invention, but which, preferably, is not a simultaneous administration as specified herein above. Preferably, sequential administration is an administration wherein administration of the pharmaceutically active compounds, preferably all pharmaceutically active compounds, starts within a time interval of 1 or 2 days, more preferably within a time interval of 12 hours, still more preferably within a time interval of 4 hours, even more preferably within a time interval of one hour, most preferably within a time interval of 5 minutes.

Preferably, the combined preparation is a pharmaceutically compatible combined preparation. The terms “pharmaceutically compatible preparation” and “pharmaceutical composition”, as used herein, relate to compositions comprising the compounds of the present invention and optionally one or more pharmaceutically acceptable carrier. The compounds of the present invention can be formulated as pharmaceutically acceptable salts. Preferred acceptable salts are acetate, methylester, HCl, sulfate, chloride and the like. The pharmaceutical compositions are, preferably, administered topically or, more preferably, systemically. Suitable routes of administration conventionally used for drug administration are oral, intravenous, or parenteral administration as well as inhalation. However, depending on the nature and mode of action of a compound, the pharmaceutical compositions may be administered by other routes as well. Moreover, the compounds can be administered in combination with other drugs either in a common pharmaceutical composition or as separated pharmaceutical compositions as specified elsewhere herein, wherein said separated pharmaceutical compositions may be provided in form of a kit of parts.

The compounds are, preferably, administered in conventional dosage forms prepared by combining the drugs with standard pharmaceutical carriers according to conventional procedures. These procedures may involve mixing, granulating and compressing or dissolving the ingredients as appropriate for the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the compound or compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Preferred doses and concentrations of the compounds of the present invention are specified elsewhere herein.

The pharmaceutical compositions and formulations referred to herein are, preferably, administered at least once in order to treat or ameliorate or prevent a disease or condition recited in this specification. However, the said pharmaceutical compositions may be administered more than one time, for example from one to four times daily up to a non-limited number of days.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The term “derivative”, as used in the context of a chemical compound of the present invention, relates to a chemical molecule having a structure related to said chemical compound of the present invention. Preferably, a derivative still has the side chains known to mediate the effect of the present invention; e.g., a derivative of 2-deoxyglucose, preferably, still comprises a glucose structure having a 2-deoxy-moiety, and, preferably, a 6-OH moiety. Preferably, a derivative can be produced from a chemical compound of the present invention by at most three, more preferably at most two, most preferably at most one chemical derivatization reactions. Preferably, the derivative is a compound which is metabolized in a mammalian, preferably a human, body into a chemical compound of the present invention. Also preferably, a derivative is a compound from which a chemical compound of the present invention can be obtained by hydrolysis.

The term “2-deoxyglucose” is understood by the skilled person and includes derivatives of the chemical compounds known under the designation, wherein the term derivative relates to chemical derivatives as specified herein below. A preferred derivative is, e.g.; 2-deoxyglucose-6-phosphate. Preferably, 2-deoxyglucose is 2-deoxy-D-glucose (CAS Registry Number: 154-17-6, (4R,5S,6R)-6-(hydroxymethyl)oxane-2,4,5-triol).

The term “flavagline”, as used herein, relates to a chemical compound comprising a cyclopenta[b]benzofuran skeleton, preferably a cyclopenta[b]tetrahydroxy-benzofuran. More preferably, the term relates to cyclopenta[b]tetrahydroxy-benzofuranes produced by or extractable from a plant from the genus Aglaia (family Meliaceae). As used in this specification, said terms include derivatives and salts of the said compounds as described herein above and below.

Preferably, the term flavagline relates to a compound of the formula (I), or a derivative thereof:

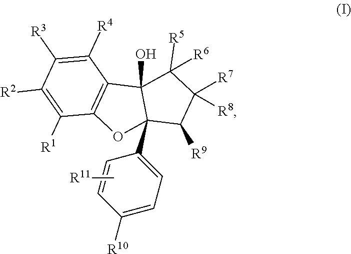

wherein
R¹ is selected from —H, halogen and alkyl;
R² is selected from alkoxy, halogen, and alkyl;
R³ is selected from —H, halogen and alkyl;
or R² and R³ together form a —O(CH₂)ₙO— unit, with n=1 or 2;
R⁴ is selected from alkoxy, halogen, and alkyl;
R⁵ is selected from hydroxyl, acyloxy, amino, monoalkylamino, dialkylamino and —NR₂—CHR₁₃—COOR₁₄, with
   R¹² being selected from —H and alkyl,
   R¹³ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
   or R¹² and R¹³ together form a —(CH₂)₃— or —(CH₂)₄— group;
   R¹⁴ being selected from alkyl and benzyl; in which case R⁶ is hydrogen,
R⁶ is selected from —H, halogen and alkyl;
or R⁵ and R⁶ together form an oxo or hydroxyimino group;
R⁷ is —H;
R⁸ is selected from —CONR¹⁶R¹⁷, —H, and —COOR¹⁵ wherein
   R¹⁵ and R¹⁶ are independently selected from methyl and —H, and
   R¹⁷ is selected from methyl, —H, 4-hydroxybutyl and 2-tetrahydrofuryl;
R⁹ is selected from phenyl which is optionally substituted, and hetaryl which is optionally substituted;
R¹⁰ is selected from alkoxy, —H, halogen, and alkyl, and
R¹¹ is selected from —H, hydroxyl, halogen, alkoxy and alkyl;
or R¹⁰ and R¹¹ are in ortho-position to each other and together form a —O(CH₂)ₙO— unit, with n=1 or 2.

The term “alkyl”, as used herein, in each case refers to an independently selected substituted or unsubstituted, linear or branched, acyclic or cyclic alkyl group, preferably an unsubstituted linear or branched acyclic alkyl group. More preferably, the term “alkyl” refers to a C₁— to C₄-alkyl group, namely methyl, ethyl, i-propyl, n-propyl, n-butyl, i-butyl, sec-butyl or tert-butyl. The above also applies when “alkyl” is used in “alkylamino” and “dialkylamino” and other terms containing the term “alkyl”.

The term “alkoxy”, as used herein, in each case refers to an independently selected substituted or unsubstituted linear or branched, acyclic or cyclic alkoxy group, preferably an unsubstituted linear or branched acyclic alkoxy group. More preferably, the term “alkoxy” refers to a C₁- to C₄-alkoxy group, namely methoxy, ethoxy, i-propyloxy, n-propyloxy, n-butyloxy, i-butyloxy, sec-butyloxy or tert-butyloxy. The above also applies when “alkoxy” is used in “thioalkoxy” and other terms containing the term “alkoxy”.

The term “acyloxy”, as used herein, in each case refers to an independently selected substituted or an unsubstituted linear or branched, acyclic or cyclic acyloxy group, preferably an unsubstituted linear or branched acyclic acyloxy group. More preferably, the term “acyloxy”, as mentioned in the above definitions of the substituents R₁ to R₁₇, in each case preferably refers to a C₁- to C₄-acyloxy group, namely formyloxy, acetoxy, i-propyloxy, n-propyloxy, n-butyloxy, i-butyloxy, sec-butyloxy or tert-butyloxy.

The term “hetaryl” as used in the above definition refers to a 5-, 6- or 7-membered carbocyclic saturated or non-saturated, aromatic or non-aromatic ring which may carry in the ring one or more heteroatoms from the group O, S, P, N.

The term “halogen” is known to the skilled person and preferably includes pseudhalogens; more preferably, the term relates to —F, —Cl, —Br, —I, —CN, or —SCN. Most preferably, the term relates to —Cl or —Br.

In a preferred embodiment, the flavagline is a chemical compound of the structure (Ia), with the definitions of substituents as specified herein above, or a derivative thereof:

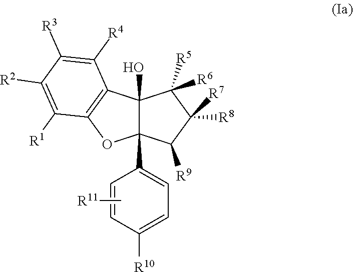

It is understood by the skilled person that formula (I) includes compounds wherein R⁶ is orientated above the plane of view and R⁵ then is orientated below the plane of view or vice versa. The same is true for R⁷ and R⁸ in formula (I), whereas in formula (Ia), R⁵ and R⁸ are orientated below the plane of view and R⁶ and R⁷ are orientated above the plane of view.

In a preferred embodiment of the present invention, the flavagline is a chemical compound of the structure (I), preferably structure (Ia), or a derivative thereof, with the following substituents:

R¹ and R³ each are hydrogen;

R² and R⁴ each are independently selected from methoxy which is optionally substituted;

R⁵ is selected from hydroxy, formyloxy and acetyloxy, alkylamino, —NR¹²—CHR¹³—COOR¹⁴, with

R¹² being selected from hydrogen and alkyl,

R¹³ being selected from: alkyl which may be substituted by: a group selected from OH, SH, alkoxy; thioalkoxy, amino, monoalkylamino, dialkylamino, carboxy, carboxyalkyl, carboxamide and guanidino groups; and phenyl and benzyl, which both may carry a substituent from the group hydroxy, indolyl and imidazolylmethyl;

R¹⁴ being selected from alkyl and benzyl;

R⁶ is hydrogen;

R⁷ is hydrogen;

R⁸ is selected from hydrogen, —COOCH₃ and CON(CH₃)₂;

R⁹ is phenyl which is optionally substituted;

R¹⁰ is methoxy;

R¹¹ is selected from hydrogen and hydroxy, or

R¹⁰ and R¹¹ are in ortho-position to each other and together form a —OCH₂O— unit.

In a more preferred embodiment of the present invention, the flavagline is a chemical compound of the structure (I), preferably structure (la), or a derivative thereof, wherein

R¹ and R³ each are hydrogen,

R² and R⁴ each are optionally substituted methoxy,

R⁵ is hydroxy or —NR¹²—CHR¹³—COOR¹⁴, with

R¹² being selected from hydrogen and alkyl,

R¹³ being selected from: alkyl which may be substituted by: a group selected from OH, SH, alkoxy; thioalkoxy, amino, monoalkylamino, dialkylamino, carboxy, carboxyalkyl, carboxamide and guanidino groups; and phenyl and benzyl, which both may carry a substituent from the group hydroxy, indolyl and imidazolylmethyl;

R¹⁴ being selected from alkyl and benzyl;

R⁶ and R⁷ each are hydrogen,

R⁸ is —CON(CH₃)₂,

R⁹ is optionally substituted phenyl,

R¹⁰ is methoxy and

R¹¹ is hydrogen;

or wherein

R¹ and R³ each are hydrogen,

R² and R⁴ each optionally substituted methoxy,

R⁵ is acetoxyor —NR¹²—CHR¹³—COOR¹⁴, with

R¹² being selected from hydrogen and alkyl,

R¹³ being selected from: alkyl which may be substituted by: a group selected from OH, SH, alkoxy; thioalkoxy, amino, monoalkylamino, dialkylamino, carboxy, carboxyalkyl, carboxamide and guanidino groups; and phenyl and benzyl, which both may carry a substituent from the group hydroxy, indolyl and imidazolylmethyl;

R¹⁴ being selected from alkyl and benzyl;

R⁶ and R⁷ each are hydrogen,

R⁸ is —CON(CH₃)₂,

R⁹ is optionally substituted phenyl,

R¹⁰ is methoxy and

R¹¹ is hydrogen;

or wherein

R¹ and R³ each are hydrogen,

R² and R⁴ each optionally substituted methoxy,

R⁵ is formyloxy or —NR¹²—CHR¹³—COOR¹⁴, with

R¹² being selected from hydrogen and alkyl,

R¹³ being selected from: alkyl which may be substituted by: a group selected from OH, SH, alkoxy; thioalkoxy, amino, monoalkylamino, dialkylamino, carboxy, carboxyalkyl, carboxamide and guanidino groups; and phenyl and benzyl, which both may carry a substituent from the group hydroxy, indolyl and imidazolylmethyl;

R¹⁴ being selected from alkyl and benzyl;

R⁶ and R⁷ each are hydrogen,

R⁸ is hydrogen or —COOCH₃,

R⁹ is optionally substituted phenyl, and

R¹⁰ and R¹¹ are in ortho-position to each other and together form a —OCH₂O— unit.

In a further embodiment of the present invention, R⁸ is a group of the formula

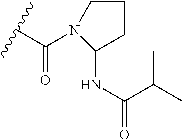 [SMILES: *C(=O)N1CCCC1NC(=O)C(C)C]

In still a further embodiment of the present invention, R⁵ and R⁸ together form a group of the formulae

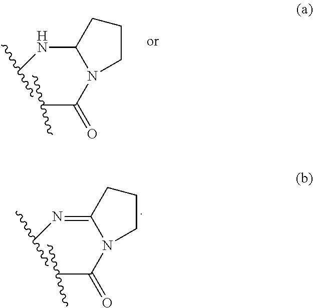 [SMILES: *NC1CCCN1C(*)=O ; */N=C1/CCCN1C(*)=O]

In a preferred embodiment, the flavagline is a chemical compound of the structure (la), or a derivative thereof,
wherein R¹ and R³ are —H, and R² and R⁴ are —O—CH₃, with the definitions of further substituents as specified for formula (I);
wherein R⁵ is hydroxyl or acyloxy, or R⁵ and R⁶ together form an oxo group, with the definitions of further substituents as specified for formula (I);
wherein R⁷ is —H and R⁸ is —COR¹⁵, with R¹⁵ being —N(CH₃)₂ or —OCH₃, with the definitions of further substituents as specified for formula (I);
wherein R⁹ is phenyl, with the definitions of further substituents as specified for formula (I); or
wherein R¹⁰ is —H and R¹¹ is alkoxy or halogen, or R¹⁰ and R¹¹ together form a —O—CH2-O— unit, with the definitions of further substituents as specified for formula (I);

In a more preferred embodiment, the flavagline is a chemical compound of the structure (Ia), or a derivative thereof,
wherein (i) R¹ and R³ are —H, R² and R⁴ are —O—CH₃, and (ii) R⁵ is hydroxyl or acyloxy, or R⁵ and R⁶ together form an oxo group, with the definitions of further substituents as specified for formula (I);
wherein (i) R¹ and R³ are —H, R² and R⁴ are —O—CH₃, and (ii) R⁷ is —H and R⁸ is —COR¹⁵, with R¹⁵ being —N(CH₃)₂ or —OCH₃, with the definitions of further substituents as specified for formula (I);
wherein (i) R¹ and R³ are —H, R² and R⁴ are —O—CH₃, and (ii) R⁹ is phenyl, with the definitions of further substituents as specified for formula (I);
wherein (i) R¹ and R³ are —H, R² and R⁴ are —O—CH₃, and (ii) R¹⁰ is —H and R¹¹ is alkoxy or halogen, or R¹⁰ and R¹¹ together form a —O—CH₂—O— unit, with the definitions of further substituents as specified for formula (I);
wherein (i) R⁵ is hydroxyl or acyloxy, or R⁵ and R⁶ together form an oxo group, and (ii) R⁷ is —H and R⁸ is —COR¹⁵, with R¹⁵ being —N(CH₃)₂ or —OCH₃, with the definitions of further substituents as specified for formula (I);
wherein (i) R⁵ is hydroxyl or acyloxy, or R⁵ and R⁶ together form an oxo group, and (ii) R⁹ is phenyl, with the definitions of further substituents as specified for formula (I);
wherein (i) R⁵ is hydroxyl or acyloxy, or R⁵ and R⁶ together form an oxo group, and (ii) R¹⁰ is —H and R¹¹ is alkoxy or halogen, or R¹⁰ and R¹¹ together form a —O—CH2-O— unit, with the definitions of further substituents as specified for formula (I);
wherein (i) R⁷ is —H and R⁸ is —COR¹⁵, with R¹⁵ being —N(CH₃)₂ or —OCH₃, and (ii) R⁹ is phenyl, with the definitions of further substituents as specified for formula (I);
wherein (i) R⁷ is —H and R⁸ is —COR¹⁵, with R¹⁵ being —N(CH₃)₂ or —OCH₃, and (ii) R¹⁰ is —H and R¹¹ is alkoxy or halogen, or R¹⁰ and R¹¹ together form a —O—CH2-O— unit, with the definitions of further substituents as specified for formula (I);

or
wherein (i) R⁹ is phenyl, and (ii) R¹⁰ is —H and R¹¹ is alkoxy or halogen, or R¹⁰ and R¹¹ together form a —O—CH2-O— unit, with the definitions of further substituents as specified for formula (I).

In an even more preferred embodiment, the flavagline is a chemical compound of the structure (Ia), or a derivative thereof,
wherein (i) R¹ and R³ are —H, R² and R⁴ are —O—CH₃, (ii) R⁵ is hydroxyl or acyloxy, or R⁵ and R⁶ together form an oxo group, and (iii) R⁷ is —H and R⁸ is —COR¹⁵, with R¹⁵ being —N(CH₃)₂ or —OCH₃, with the definitions of further substituents as specified for formula (I);
wherein (i) R¹ and R³ are —H, R² and R⁴ are —O—CH₃, (ii) R⁵ is hydroxyl or acyloxy, or R⁵ and R⁶ together form an oxo group, and (iii) R⁹ is phenyl, with the definitions of further substituents as specified for formula (I);
wherein (i) R¹ and R³ are —H, R² and R⁴ are —O—CH₃, (ii) R⁵ is hydroxyl or acyloxy, or R⁵ and R⁶ together form an oxo group, and (iii) R¹⁰ is —H and R¹¹ is alkoxy or halogen, or R¹⁰ and R¹¹ together form a —O—CH2-O— unit, with the definitions of further substituents as specified for formula (I);
wherein (i) R¹ and R³ are —H, R² and R⁴ are —O—CH₃, (ii) R⁷ is —H and R⁸ is —COR¹⁵, with R¹⁵ being —N(CH₃)₂ or —OCH₃, and (iii) R⁹ is phenyl, with the definitions of further substituents as specified for formula (I);
wherein (i) R¹ and R³ are —H, R² and R⁴ are —O—CH₃, (ii) R⁷ is —H and R⁸ is —COR¹⁵, with R¹⁵ being —N(CH₃)₂ or —OCH₃, and (iii) R¹⁰ is —H and R¹¹ is alkoxy or halogen, or R¹⁰ and R¹¹ together form a —O—CH2-O— unit, with the definitions of further substituents as specified for formula (I);
wherein (i) R¹ and R³ are —H, R² and R⁴ are —O—CH₃, (ii) R⁹ is phenyl, and (iii) R¹⁰ is —H and R¹¹ is alkoxy or halogen, or R¹⁰ and R¹¹ together form a —O—CH2-O— unit, with the definitions of further substituents as specified for formula (I);
wherein (i) R⁵ is hydroxyl or acyloxy, or R⁵ and R⁶ together form an oxo group, (ii) R⁷ is —H and R⁸ is —COR¹⁵, with R¹⁵ being —N(CH₃)₂ or —OCH₃, and (iii) R⁹ is phenyl, with the definitions of further substituents as specified for formula (I);
wherein (i) R⁵ is hydroxyl or acyloxy, or R⁵ and R⁶ together form an oxo group, and (ii) R⁷ is —H and R⁸ is —COR¹⁵, with R¹⁵ being —N(CH₃)₂ or —OCH₃, and (iii) R¹⁰ is —H and R¹¹ is alkoxy or halogen, or R¹⁰ and R¹¹ together form a —O—CH2-O— unit, with the definitions of further substituents as specified for formula (I);
wherein (i) R⁵ is hydroxyl or acyloxy, or R⁵ and R⁶ together form an oxo group, (ii) R⁹ is phenyl, and (iii) R¹⁰ is —H and R¹¹ is alkoxy or halogen, or R¹⁰ and R¹¹ together form a —O—CH2-O— unit, with the definitions of further substituents as specified for formula (I); or
wherein (i) R⁷ is —H and R⁸ is —COR¹⁵, with R¹⁵ being —N(CH₃)₂ or —OCH₃, (ii) R⁹ is phenyl, and (iii) R¹⁰ is —H and R¹¹ is alkoxy or halogen, or R¹⁰ and R¹¹ together form a —O—CH2-O— unit, with the definitions of further substituents as specified for formula (I).

In a still more preferred embodiment, the flavagline is a chemical compound of the structure (Ia), or a derivative thereof,
wherein (i) R¹ and R³ are —H, R² and R⁴ are —O—CH₃, (ii) R⁵ is hydroxyl or acyloxy, or R⁵ and R⁶ together form an oxo group, (iii) R⁷ is —H and R⁸ is —COR¹⁵, with R¹⁵ being —N(CH₃)₂ or —OCH₃, and (iv) R⁹ is phenyl, with the definitions of further substituents as specified for formula (I);
wherein (i) R¹ and R³ are —H, R² and R⁴ are —O—CH₃, (ii) R⁵ is hydroxyl or acyloxy, or R⁵ and R⁶ together form an oxo group, (iii) R⁷ is —H and R⁸ is —COR¹⁵, with R¹⁵ being —N(CH₃)₂ or —OCH₃, and (iv) R¹⁰ is —H and R¹¹ is alkoxy or halogen, or R¹⁰ and R¹¹ together form a —O—CH2-O— unit, with the definitions of further substituents as specified for formula (I);
wherein (i) R¹ and R³ are —H, R² and R⁴ are —O—CH₃, (ii) R⁵ is hydroxyl or acyloxy, or R⁵ and R⁶ together form an oxo group, (iii) R⁹ is phenyl, and (iv) R¹⁰ is —H and R¹¹ is alkoxy or halogen, or R¹⁰ and R¹¹ together form a —O—CH2-O— unit, with the definitions of further substituents as specified for formula (I);
wherein (i) R¹ and R³ are —H, R² and R⁴ are —O—CH₃, (ii) R⁷ is —H and R⁸ is —COR¹⁵, with R¹⁵ being —N(CH₃)₂ or —OCH₃, (iii) R⁹ is phenyl, and (iv) R¹⁰ is —H and R¹¹ is alkoxy or halogen, or R¹⁰ and R¹¹ together form a —O—CH2-O— unit, with the definitions of further substituents as specified for formula (I);

or
wherein (i) R⁵ is hydroxyl or acyloxy, or R⁵ and R⁶ together form an oxo group, (ii) R⁷ is —H and R⁸ is —COR¹⁵, with R¹⁵ being —N(CH₃)₂ or —OCH₃, (iii) R⁹ is phenyl, and (iv) R¹⁰ is —H and R¹¹ is alkoxy or halogen, or R¹⁰ and R¹¹ together form a —O—CH2-O— unit, with the definitions of further substituents as specified for formula (I);

In a most preferred embodiment, the flavagline is a chemical compound of the structure (Ia), or a derivative thereof,
wherein (i) R¹ and R³ are —H, R² and R⁴ are —O—CH₃, (ii) R⁵ is hydroxyl or acyloxy, or R⁵ and R⁶ together form an oxo group, (iii) R⁷ is —H and R⁸ is —COR¹⁵, with R¹⁵ being —N(CH₃)₂ or —OCH₃, (iv) R⁹ is phenyl, and (v) R¹⁰ is —H and R¹¹ is alkoxy or halogen, or R¹⁰ and R¹¹ together form a —O—CH2-O— unit.

Preferably, the term flavagline relates to a compound selected from the group consisting of rocaglamide, aglaroxin C, cyclorocaglamide, rocaglaol, methylrocaglate (aglafolin), desmethylrocaglamide, pannellin and the recently isolated dioxanyloxy-modified derivatives silvestrol and episilvestrol (Hwang et al., 2004, J. Org. Chem. Vol. 69: pages 3350-3358). It is understood by the skilled person that the term “rocaglamide” is a generic term for a compound of formula (II) (named Rocaglamide A or Roc-A in the example section), formula (III), formula (IV), formula (V) (named Rocaglamide Q or Roc-Q in the example section), formula (VI) (referred to as Rocaglamide AR or Roc-AR in the present application), formula (VII) (known as Rocaglamide U or Roc-U), and formula (VIII) (known as Rocaglamide W or Roc-W). More preferably, the flavagline is Rocaglamide Q or Rocaglamide AR; most preferably, the flavagline is Rocaglamide A ((1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide). Preferably, the flavagline is a compound of formula (IX), known as FL3 (1R,3S,3aR,8bS)-3a-(4-Bromophenyl)-6,8-dimethoxy-3-phenyl-2,3,3a,8b-tetrahydro-1H-cyclopenta[b]benzofuran-1,8b-diol), or a derivative thereof.

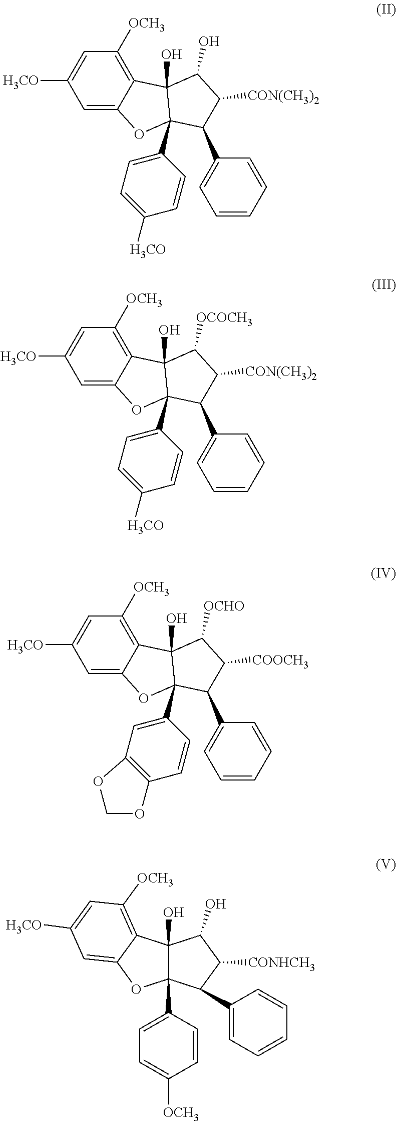

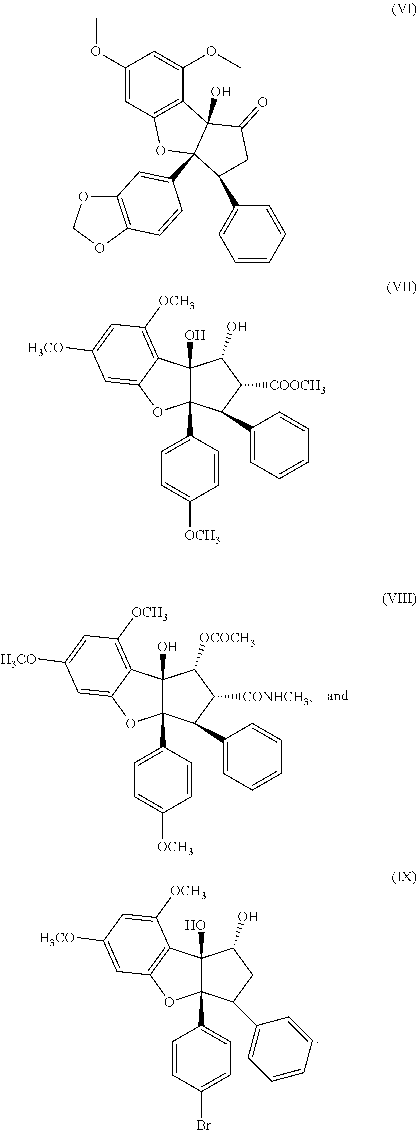

Most preferably, the flavagline is Rocaglamide A or FL3, as specified herein above.

Advantageously, it was found during the work underlying the present invention, that in a combined treatment of cancer cells with a flavagline and 2-deoxyglucose, the cancer cells are sensitized to 2-deoxyglucose. Accordingly, 2-deoxyglucose may be used at a lower concentration in the presence of a flavagline, while still having the same effect on cancer cells. The sensitization is to such an extent that a 2-deoxyglucose concentration may be used in cancer treatment which has been shown to cause no or at least less severe adverse reactions in humans. Similarly, the sensitivity of cancer cells to flavaglines was increased in the presence of 2-deoxyglucose. Importantly, it was found that normal cells are protected from the effect of flavaglines by the presence of 2-deoxyglucose.

The definitions made above apply mutatis mutandis to the following. Additional definitions and explanations made further below also apply for all embodiments described in this specification mutatis mutandis.

The present invention further relates to a combined preparation according to the present invention for use as a medicament. Moreover, the present invention relates to a combined preparation according to the present invention for use in the treatment of cancer.

In the medical uses according to the present invention, 2-deoxyglucose, preferably, is administered at a concentration causing at most tolerable adverse drug reactions. The term “tolerable adverse drug reactions”, preferably, relates to at most grade 3, more preferably at most grade 2, adverse events according to the Common Terminology Criteria for Adverse Events v4.0 (CTCAE), published by the U.S. Department of Health and Human Services on May 28, 2009. More preferably, the term relates to gastrointestinal bleeding in at most 25% of patients receiving treatment, even more preferably in at most 10% of patients receiving treatment and/or reversible grade 3 prolongation of the interval between start of the Q wave and the end of the T wave in the electrical cycle of the heart (QTc prolongation) in at most 50% of patients receiving treatment, preferably at most 25% of patients receiving treatment. Preferably, 2-deoxyglucose is administered at a concentration of from 0.01 mM to 20 mM, more preferably, of from 0.1 mM to 4 mM, still more preferably of from about 0.25 to about 1 mM, even more preferably of from 0.5 mM to 1 mM, most preferably of about 1 mM. Preferably, 2-deoxyglucose is administered at a daily dose of from 10 mg/kg to 150 mg/kg, more preferably of from 25 to 125 mg/kg, even more preferably, of from 50 mg/kg to 100 mg/kg, still more preferably of from 70 mg/kg to 90 mg/kg, most preferably, of from 75 mg/kg to 80 mg/kg; preferably, at most grade 3 adverse events are observed with these doses.

Also preferably, 2-deoxyglucose is administered at a daily dose of from 10 mg/kg to 100 mg/kg, more preferably of from 25 to 90 mg/kg, even more preferably, of from 30 mg/kg to 85 mg/kg, still more preferably of from 35 mg/kg to 65 mg/kg, most preferably, of from 40 mg/kg to 55 mg/kg; preferably, at most grade 2 adverse events are observed with these doses. Also preferably, 2-deoxyglucose is administered at a three times per week dose or, preferably, weekly dose, of from 50 mg/kg to 1000 mg/kg, more preferably of from 75 to 750 mg/kg, even more preferably, of from 100 mg/kg to 600 mg/kg, still more preferably of from 125 mg/kg to 500 mg/kg, most preferably, of from 150 mg/kg to 250 mg/kg. Also preferably, 2-deoxyglucose is administered at a once per month dose of from 200 mg/kg to 2000 mg/kg, more preferably of from 400 to 1500 mg/kg, even more preferably, of from 425 mg/kg to 1250 mg/kg, still more preferably of from 450 mg/kg to 1000 mg/kg, most preferably, of from 475 mg/kg to 700 mg/kg. It is understood by the skilled person that the above administration of 2DG may comprise treatment free intervals to allow for recovery of the patient, e.g. two weeks with daily dose, followed by one week without administration of 2DG.

Moreover, in the medical uses according to the present invention, the flavagline or flavaglines is/are, preferably, administered at a concentration of from 0.02 μM to 1 mM, more preferably, of from 0.02 μM to 250 μM, still more preferably of from 0.02 μM to 25 μM, most preferably of from 0.025 μM to 0.1 μM. Preferably, the flavagline or flavaglines is/are administered at a daily dose of, preferably, from 0.01 mg/kg to 500 mg/kg, more preferably from 0.01 mg/kg to 125 mg/kg, still more preferably from 0.01 mg/kg to 12.5 mg/kg, most preferably of about 0.0125 mg/kg to 0.05 mg/kg. Preferably, if more than one flavagline is administered, the aforementioned concentration or dose is the sum of the concentrations of the flavaglines administered.

Preferably, the combined preparation is for use in the treatment of cancer. The term “cancer”, as used herein, refers to a disease of an animal, preferably man, characterized by uncontrolled growth by a group of body cells (“cancer cells”). This uncontrolled growth may be accompanied by intrusion into and destruction of surrounding tissue and possibly spread of cancer cells to other locations in the body.

Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, Burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, gallbladder cancer, gastric cancer, gastrointestinal stromal tumor, gestational trophoblastic tumor, hairy cell leukemia, head and neck cancer, hepatocellular cancer, Hodgkin lymphoma, hypopharyngeal cancer, hypothalamic and visual pathway glioma, intraocular melanoma, Kaposi sarcoma, laryngeal cancer, medulloblastoma, medulloepithelioma, melanoma, Merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, Waldenström macroglobulinemia, and wilms tumor.

More preferably, the cancer is leukemia, lymphoma, HPV-related cancer, colorectal carcinoma, gastric cancer, pancreas cancer, lung cancer, brain cancer, or breast cancer. A preferred HPV-related cancer is cervix carcinoma. A preferred colorectal carcinoma is colon carcinoma. Preferred lung cancers are small cell lung cancer and non-small cell lung cancer. Preferred brain cancers are glioma, astrocytoma, and glioblastoma.

The present invention also relates to a flavagline for use in a combination therapy against cancer comprising administration of 2-deoxyglucose. Moreover, the present invention relates to 2-deoxyglucose for use in a combination therapy against cancer comprising administration of a flavagline.

The term “combination therapy” is understood by the skilled person and, preferably, relates to a treatment of a subject including administration of at least two modes of treatment and/or at least two pharmaceutically active compounds, with “mode of treatment” relating to the scientific principle underlying the treatment, e.g. surgery, radiation therapy, and administration of pharmaceutically active compounds. Accordingly, the combination therapy according to the present specification, preferably, comprises administration of a flavagline and 2-deoxyglucose. It is understood that the combination therapy, preferably, may comprise further steps, including administration of further modes of treatment and/or further pharmaceutically active compounds, e.g. chemotherapeutic agents.

The term “subject”, as referred to herein, encompasses animals, preferably mammals, more preferably, humans. Preferably, the subject suffers from, is suspected to suffer from, or is at risk to suffer from a cancer as specified elsewhere herein. Subjects which suffer from the said disease(s) can be identified by the accompanying symptoms known for the disease(s). These symptoms are known in the art and described, e.g., in medical textbooks. A subject suspected to suffer from the aforementioned disease(s) may be any apparently healthy subject, e.g., investigated by routine clinical screening, or may be a subject being at risk for developing the aforementioned disease.

Furthermore, the present invention relates to a method of treating cancer in a subject afflicted with cancer, comprising administering a flavagline and 2-deoxyglucose to said subject, thereby treating said cancer.

The method of the present invention, preferably, is an in vivo method. Moreover, it may comprise steps in addition to those explicitly mentioned above. For example, further steps may relate, e.g., to surgically removing tumor tissue before or after administration of said pharmaceutically active compounds, or surveying heart function of said subject. Moreover, one or more of said steps may be performed by automated equipment. Preferably, in the method of treating cancer according to the present specification, said flavagline and said 2-deoxyglucose are administered simultaneously.

Moreover, the present invention relates to the use of 2-deoxyglucose and of a flavagline for the manufacture of a pharmaceutical composition for the treatment of cancer.

Preferably, the pharmaceutical composition is a combined preparation as specified elsewhere herein. Preferably, the pharmaceutical composition is a composition comprising a mixture of said 2-deoxyglucose and said flavagline, i.e., preferably, the pharmaceutical composition is a mixed formulation as specified elsewhere herein.

The present invention also relates to a use of a combined preparation for combined or separate and/or for simultaneous or sequential use comprising 2-deoxyglucose and a flavagline for treating cancer.

Further, the present invention relates to a process for the preparation of a combined preparation, wherein said combined preparation is a preparation comprising a mixture of flavagline and 2-deoxyglucose, comprising the step of mixing a flavagline and 2-deoxyglucose. Thus, preferably, the process is a process for the preparation of a mixed formulation of a flavagline and 2-deoxyglucose.

The process of the present invention, preferably, may comprise steps in addition to those explicitly mentioned above. For example, further steps may relate, e.g., to providing a flavagline and/or 2-deoxyglucose in a pharmaceutically acceptable form or formulating the mixture obtained as a pharmaceutical composition.

In view of the above, the following embodiments are preferred:

### EMBODIMENT 1

A combined preparation comprising 2-deoxyglucose and a flavagline.

### EMBODIMENT 2

The combined preparation of embodiment 1, wherein said flavagline is a compound of the formula (I),

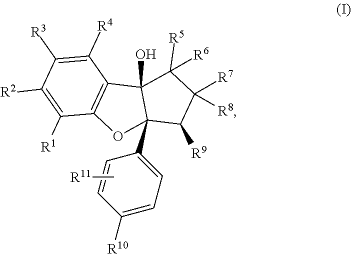

or, preferably, of the formula (Ia)

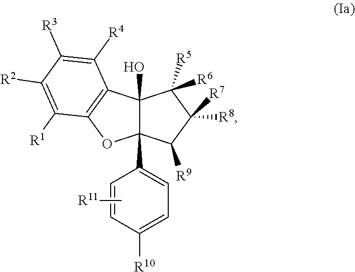

wherein
R¹ is selected from —H, halogen and alkyl;
R² is selected from alkoxy, halogen, and alkyl;
R³ is selected from —H, halogen and alkyl;
   or R² and R³ together form a —O(CH₂)ₙO— unit, with n=1 or 2;
R⁴ is selected from alkoxy, halogen, and alkyl;
R⁵ is selected from hydroxyl, acyloxy, amino, monoalkylamino, dialkylamino and —NR₁₂—CHR₁₃—COOR₁₄, with
   R¹² being selected from —H and alkyl,
   R¹³ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
   or R¹² and R¹³ together form a —(CH₂)₃— or —(CH₂)₄— group;
   R¹⁴ being selected from alkyl and benzyl; in which case R⁶ is hydrogen,
R⁶ is selected from —H, halogen and alkyl;
   or R⁵ and R⁶ together form an oxo or hydroxyimino group;
R⁷ is —H;
R⁵ is selected from —CONR¹⁶R¹⁷, —H, and —COOR¹⁵ wherein
   R¹⁵ and R¹¹ are independently selected from methyl and —H, and
   R¹⁷ is selected from methyl, —H, 4-hydroxybutyl and 2-tetrahydrofuryl;
R⁹ is selected from phenyl which is optionally substituted, and hetaryl which is optionally substituted;
R¹⁰ is selected from alkoxy, —H, halogen, and alkyl, and
R¹¹ is selected from —H, hydroxyl, halogen, alkoxy and alkyl;
   or R¹⁰ and R¹¹ are in ortho-position to each other and together form a —O(CH₂)ₙO— unit, with n=1 or 2,
or a derivative or a salt thereof.

### EMBODIMENT 3

The combined preparation embodiment 1 or 2, wherein said flavagline is a compound comprising a structure selected from the list consisting of formulas (II) to (IX):

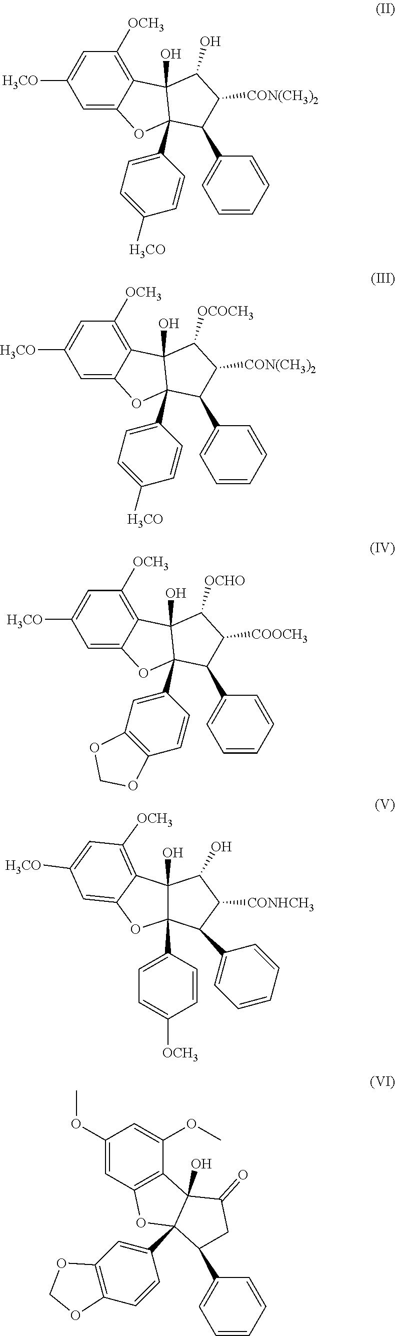

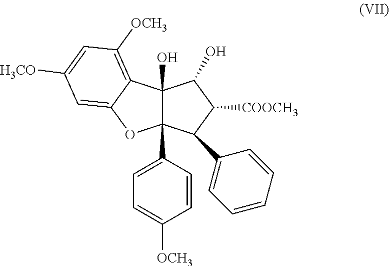 [SMILES: COC(=O)[C@H]1[C@@H](O)[C@@]2(O)c3c(OC)cc(OC)cc3O[C@@]2(c2ccc(OC)cc2)[C@@H]1c1ccccc1]

### EMBODIMENT 4

The combined preparation according to any one of embodiments 1 to 3, wherein said flavagline is (1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide (Rocaglamide A) or a derivative thereof; or (1R,3S,3aR,8bS)-3a-(4-Bromophenyl)-6,8-dimethoxy-3-phenyl-2,3,3a,8b-tetrahydro-1H-cyclopenta[b]benzofuran-1,8b-diol (FL3) or a derivative thereof.

### EMBODIMENT 5

The combined preparation of any one of embodiments 1 to 4, wherein said 2-deoxyglucose is 2-Deoxy-D-glucose ((4R,5S,6R)-6-(hydroxymethyl)oxane-2,4,5-triol).

### EMBODIMENT 6

The combined preparation of any one of embodiments 1 to 5, wherein said combined preparation is a preparation comprising a mixture of said flavagline and said 2-deoxyglucose.

### EMBODIMENT 7

The combined preparation of any one of embodiments 1 to 6, wherein said combined preparation is for combined or separate and/or for simultaneous or sequential use.

### EMBODIMENT 8

The combined preparation of any one of embodiments 1 to 7, wherein said combined preparation is a pharmaceutically compatible preparation.

### EMBODIMENT 9

A combined preparation according to any one of embodiments 1 to 8 for use as a medicament.

### EMBODIMENT 10

A combined preparation according to any one of embodiments 1 to 8 for use in the treatment of cancer.

### EMBODIMENT 11

The combined preparation for use of embodiment 10, wherein said cancer is leukemia, lymphoma, HPV-related cancer, colorectal carcinoma, gastric cancer, pancreas cancer, lung cancer, brain cancer, or breast cancer, preferably, wherein said cancer is cervix carcinoma, colon carcinoma, small cell lung cancer, non-small cell lung cancer, glioma, astrocytoma, or glioblastoma.

### EMBODIMENT 12

The combined preparation for use of embodiment 10 or 11, wherein said 2-deoxyglucose is administered at a concentration causing at most tolerable adverse drug reactions.

### EMBODIMENT 13

The combined preparation for use of any one of embodiments 10 to 12, wherein said 2-deoxyglucose is administered at a concentration of from 0.01 mM to 20 mM, preferably, of from 0.1 mM to 4 mM, more preferably of about 1 mM.

### EMBODIMENT 14

A flavagline for use in a combination therapy against cancer comprising administration of 2-deoxyglucose.

### EMBODIMENT 15

The flavagline for use of embodiment 14, wherein said cancer is leukemia, lymphoma, HPV-related cancer, colorectal carcinoma, gastric cancer, pancreas cancer, lung cancer, brain cancer, or breast cancer, preferably, wherein said cancer is cervix carcinoma, colon carcinoma, small cell lung cancer, non-small cell lung cancer, glioma, astrocytoma, or glioblastoma.

### EMBODIMENT 16

2-deoxyglucose for use in a combination therapy against cancer comprising administration of a flavagline.

### EMBODIMENT 17

The 2-deoxyglucose for use of embodiment 16, wherein said cancer is leukemia, lymphoma, HPV-related cancer, colorectal carcinoma, gastric cancer, pancreas cancer, lung cancer, brain cancer, or breast cancer, preferably, wherein said cancer is cervix carcinoma, colon carcinoma, small cell lung cancer, non-small cell lung cancer, glioma, astrocytoma, or glioblastoma.

### EMBODIMENT 18

A method of treating cancer in a subject afflicted with cancer, comprising administering a flavagline and 2-deoxyglucose to said subject, thereby treating said cancer.

### EMBODIMENT 19

The method of treating cancer of embodiment 18, wherein said flavagline and said 2-deoxyglucose are administered simultaneously.

### EMBODIMENT 20

Use of 2-deoxyglucose and of a flavagline for the manufacture of a pharmaceutical composition for the treatment of cancer.

### EMBODIMENT 21

The use of embodiment 20, wherein said pharmaceutical composition is a composition comprising a mixture of said 2-deoxyglucose and said flavagline.

### EMBODIMENT 22

Use of a combined preparation for combined or separate and/or for simultaneous or sequential use comprising 2-deoxyglucose and a flavagline for treating cancer.

### EMBODIMENT 23

A process for the preparation of a combined preparation according to embodiment 6, comprising the step of mixing a flavagline and 2-deoxyglucose.

### EMBODIMENT 24

The process of embodiment 23, further comprising the step of formulating the mixture of a flavagline and 2-deoxyglucose as a pharmaceutical composition.

All references cited in this specification are herewith incorporated by reference with respect to their entire disclosure content and the disclosure content specifically mentioned in this specification.

### FIGURE LEGENDS

FIG. 1: Determination of IC₅₀ of Rocaglamide and FL3. Treatment schemes are illustrated by different line patterns explained on the right side of each graphic. Solid lines with squares represent treatment with Rocaglamide, dotted lines with circles represent treatment with Rocaglamide and 2DG in combination, dotted lines with triangles represent treatment with FL3, and dotted lines with rhombus represent treatment with FL3 and 2DG in combination. X axis represents treatment doses of flavaglines (Rocaglamide or FL3), y axis represents relative cell viability (determined by MT assay) in %; cells not treated with flavaglines are used as a reference (corresponding to 100% viability). A. HCT116 cells. B. HT29 cells. C: normal human PBMCs.

FIG. 2: Tumor volume in mice injected with HCT116 cells (10⁷ cells) and treated with drugs daily by intraperitoneal injection. A. Mice were treated with 200 mg/kg 2-DG or with 0.5 mg/kg Rocaglamide or with a combination of said compounds for 10 days after tumor size reached about 15-40 mm³. Data representing the tumor volume at the end of the experiment are shown, each triangle representing tumor volume of one mouse. B. Data from (A) presented as bar graphs.

FIG. 3: Cell viability of U136MG cells treated with 2DG (1 mM or 4 mM) and of PBMCs treated with 2DG (1 mM) alone or in combination with Rocaglamide (0.1 μM, 0.025 μM, 0.01 μM). The results demonstrate that 4 mM 2DG leads to a reduction of U136MG cell viability to 46% compared to the untreated control. Combination with Rocaglamide allows a significant dose reduction of 2DG to a dose of 1 mM: In combination with 0.1 μM Rocaglamide, 1 mM 2DG leads to a significantly higher reduction of tumor cell viability than 4 mM 2DG alone (4 mM 2DG, 46% cell viability compared to 1 mM 2DG+0.1 μM Rocaglamide, 37% cell viability, p=0.013). In combination with 0.025 μM Rocaglamide, 1 mM 2DG leads to a significantly higher reduction of tumor cell viability than 4 mM 2DG alone (4 mM 2DG, 46% cell viability compared to 1 mM 2DG+0.025 μM Rocaglamide, 38% cell viability, p=0.014). In contrast, cell viability of PBMC was reduced to 81.7% of the untreated control cells upon 1 mM 2DG treatment, and only a minor effect of additional Rocaglamide treatment was observed.

The following Examples shall merely illustrate the invention. They shall not be construed, whatsoever, to limit the scope of the invention.

### EXAMPLE 1: SENSITIZATION OF TUMOR CELLS TOWARDS 2-DG USING ROCAGLAMIDE IN VITRO

Methods:

The MTT (MethylThiazol Tetrazolium) assay was used to determine cell viability. The MTT assay is a colorimetric method for measuring the activity of cellular enzymes that reduce the tetrazolium dye, MTT, to its insoluble formazan, giving a purple color. This method was used in the present experiments to reflect the number of viable cells present and to measure cytotoxicity (loss of viable cells).

Cells were plated in flat-bottom 96-well plates at a cell number of 10,000 in 100 p of medium per well. RPMI 1640 (Roswell Park Memorial Institute, Gibco, City, State) supplemented with fetal bovine serum (10%) and penicillin/streptomycin (1%) medium was used for cell culturing and plating. After plating, cells were incubated at 37° C. in an incubator at 5% CO2 concentration for 24 hours before the beginning of treatment. After that, when cells became adherent, the medium was removed and 2-DG and rocaglamide were added to the cells at a concentration of 1 mM and 0.1 μM, respectively. After 48 hours of incubation, 20 μl of MTT reagent was added to each well. Cells were incubated for 1 hour at 37° C. before colorimetric assessment

To determine the amount of viable cells after the treatment, the absorption was measured at a wave length of 485 nm with the help of an ELISA reader device (TECAN GENios, Austria). All measurements were carried out in quadruplicate. Mean values and standard deviations were calculated from the measured values after background subtraction. As background, absorption was measured in wells containing medium alone.

Results:

2-DG treatment at a concentration of 1 mM induced a significant response (reduction of viability to less than 80% of the untreated cells) in 9 out of 15 cell lines analyzed. Sensitization by concomitant addition of Rocaglamide dramatically enhanced the observed responses, as all 13 out of 13 cell lines showed a significant response after 2-DG+rocaglamide treatment. Moreover, the observed responses were significantly more pronounced upon sensitization with Rocaglamide (Table 1).

TABLE 1
Cell viability after treatment of tumor cell lines with 2-deoxyglucose
or/and rocaglamide. Results are shown for 2-DG treatment alone
(1 mM, “2DG”), rocaglamide treatment alone (0.1 μM,
“Rocaglamide”), 2-DG + rocaglamide (1 mM,
0.1 μM, respectively, “2DG + Roc”).

| Cell line | 2DG | Rocaglamide | 2DG + Roc |
|---|---|---|---|
| SMA560 | 102.4 | 92.8 | 25.5 |
| LNT229 | 112.7 | 100.5 | 58.7 |
| C3 | 102.6 | 45.3 | 26.8 |
| U373 | 95.6 | 76.4 | 53.9 |
| RKO | 118.4 | 42.9 | 38.7 |
| K073 | 89.6 | 35.9 | 21.3 |
| HT29 | 87.5 | 52.3 | 37 |
| NCH82 | 68.3 | 63.2 | 35.8 |
| H146 | 90.5 | 47.3 | 39.2 |
| NCH89 | 83.7 | 56.6 | 44.1 |
| GL261 | 63.8 | 58.2 | 35.2 |
| SW480 | 70.8 | 59.5 | 40.1 |
| HCT116 | 82 | 14.8 | 11.3 |

### EXAMPLE 2: ALTERATION OF THE IC₅₀ OF FLAVAGLINES BY COMBINATION WITH 2DG

Methods:

The MTT (MethylThiazol Tetrazolium) assay was the same as in Example 1. Colorectal cancer cells (HCT116 and HT29) as well as normal human PBMCs were plated in flat-bottom 96-well plates at a cell number of 10,000 and 100,000, respectively, in 100 μl of medium per well. DMEM (Roswell Park Memorial Institute, Gibco, Paisley, UK) supplemented with 10% fetal bovine serum and 1% penicillin/streptomycin (Gibco, N.Y., USA) medium was used for cell culturing and plating of tumor cells. Medium for PBMCs consisting of Iscove's modified DMEM (IMDM) supplemented by 10% human AB serum, 1% L-glutamin and 0,05% gentamycin was used for plating PBMCs. After plating, rocaglamide and FL3 were applied at five increasing concentrations (10, 25, 50, 75 and 100 nM) as single substances and in combination with 1 mM 2DG. Cells were then incubated with drugs in a CO₂ chamber at 5% CO₂ concentration (Memmert Incubator, Büchenbach, Germany) for 48 hours. Afterwards, 20 μl of MTT reagent (Promega, Madison, Wis., USA) were added to each well. Cells were incubated for 1 hour at 37° C. as above. To determine the amount of viable cells after the treatment the absorption was measured at a wave length of 485 nm in ELISA reader device (TECAN GENios, Salzburg, Austria). All measurements were carried out in quadruplicates.

Mean values and standard deviations were calculated from the measured values after background subtraction. As background, absorption was measured in wells containing only medium.

Results:

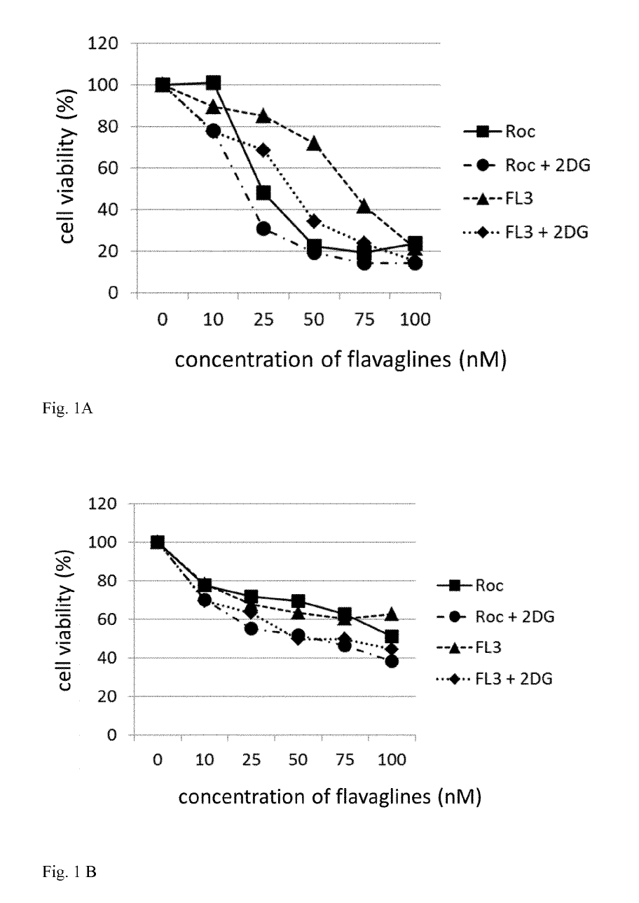

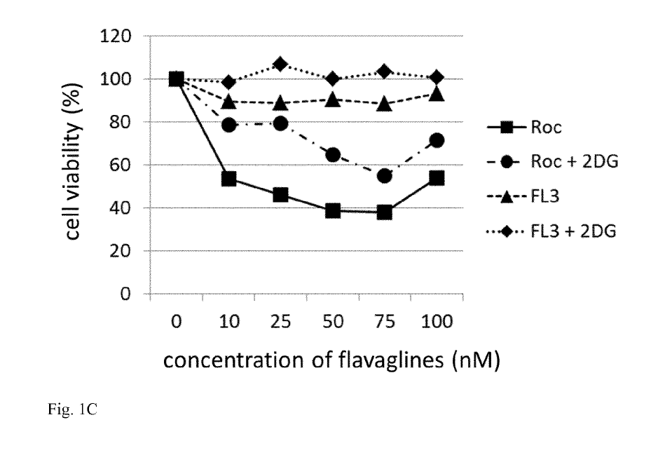

Application of Rocaglamide or FL3 in combination with 2DG on tumor cells substantially reduced the IC₅₀ of both drugs (FIGS. 1A and B). In contrast, the IC₅₀ of both drugs for normal cells was increased in the presence of 2DG (FIG. 1C). This example demonstrates that application of 2DG in combination with Rocaglamide or FL3 substantially increases the sensitivity of tumor cells towards these drugs and simultaneously dramatically reduces their effect on cell viability of normal cells.

### EXAMPLE 3: INHIBITION OF TUMOR GROWTH IN MICE BY 2-DG AND ROCAGLAMIDE

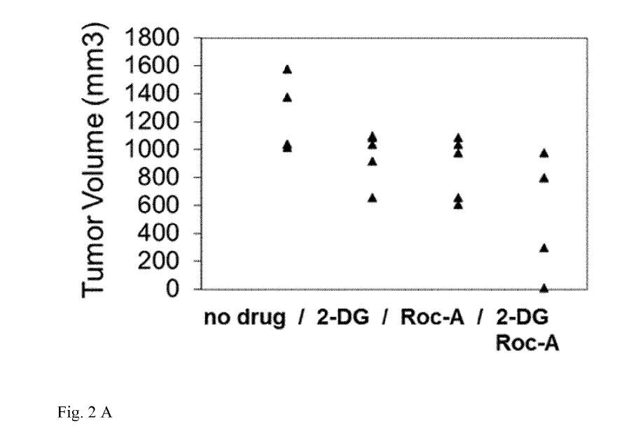

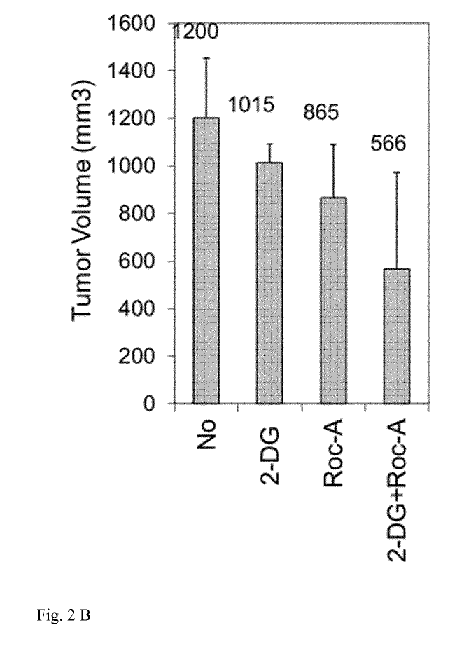

HCT116 cells (2×10⁶) were subcutaneously injected in immunodeficient mice. Treatment with 2-DG (200 mg/kg body weight) alone, Rocaglamide (0.5 mg/kg) alone, or the combination of 2-DG and Rocaglamide was started on day 6 after tumor cell injection. Combination therapy led to a significant reduction of tumor growth, and mice of the combination therapy group showed lowest tumor volume compared to individual therapy or the mock-injected control group. The results of the animal experiments are shown in FIG. 2.

### EXAMPLE 4: REDUCTION OF 2DG DOSE IN COMBINATION THERAPY WITH ROCAGLAMIDE

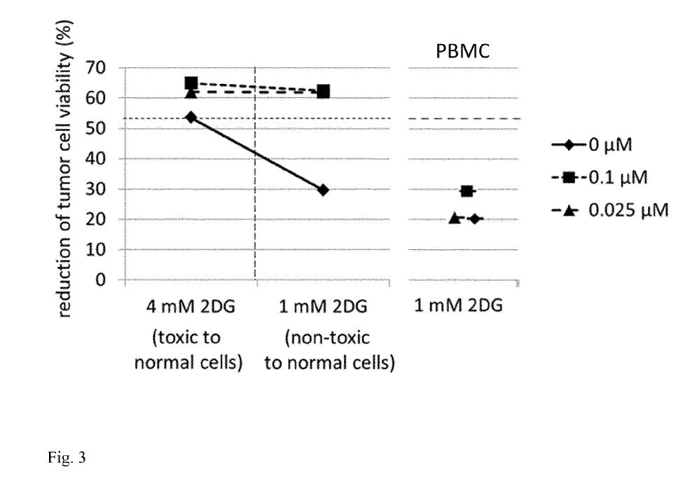

In order to evaluate the potential of Rocaglamide to reduce the dose of 2DG while at the same time maintaining its antitumoral effect, combinations of different doses of 2DG with Rocaglamide were evaluated in U138MG cells (mouse glioma). The cell-killing effect of the combination of Rocaglamide and 2DG was then compared with the effect of 2DG alone. The results demonstrated that combination of 1 mM 2DG already with low doses (0.025 μM) of Rocaglamide allows to achieve a similar killing effect to the one observed with 4 mM 2DG alone (FIG. 3A), i.e. in combination therapy with Rocaglamide, an at least fourfold dose reduction is feasible. As shown in FIG. 3B, the results demonstrate that 4 mM 2DG leads to a 54% reduction of cell viability compared to the untreated control. Combination with Rocaglamide allows a significant dose reduction of 2DG to a dose of 1 mM: In combination with 0.1 μM Rocaglamide, 1 mM 2DG leads to a significantly higher reduction of tumor cell viability than 4 mM 2DG alone (4 mM 2DG, 54% reduction of cell viability compared to 1 mM 2DG&0.1 μM Rocaglamide, 63% cell viability, p=0.013). In combination with 0.025 μM Rocaglamide, 1 mM 2DG leads to a significantly higher reduction of tumor cell viability than 4 mM 2DG alone (4 mM 2DG, 54% reduction of cell viability compared to 1 mM 2DG&0.025 μM Rocaglamide, 62% reduction of cell viability, p=0.014).

1-15. (canceled)

16. A combined preparation comprising 2-deoxyglucose and a flavagline.

17. The combined preparation of claim 16, wherein said flavagline is a compound of the formula (I), 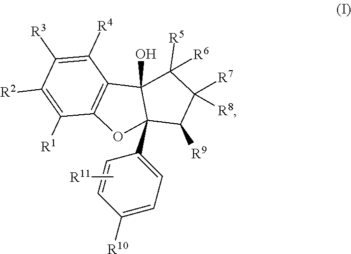
wherein
R¹ is selected from —H, halogen and alkyl;
R² is selected from alkoxy, halogen, and alkyl;
R³ is selected from —H, halogen and alkyl;
or R² and R³ together form a —O(CH₂)ₙO— unit, with n=1 or 2;
R⁴ is selected from alkoxy, halogen, and alkyl;
R⁵ is selected from hydroxyl, acyloxy, amino, monoalkylamino, dialkylamino and —NR₁₂—CHR₁₃—COOR₁₄, with
R¹² being selected from —H and alkyl,
R¹³ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
or R¹² and R¹³ together form a —(CH₂)₃— or —(CH₂)₄— group;
R¹⁴ being selected from alkyl and benzyl; in which case R⁶ is hydrogen,
R⁶ is selected from —H, halogen and alkyl;
or R⁵ and R⁶ together form an oxo or hydroxyimino group;
R⁷ is —H;
R⁸ is selected from —CONR¹⁶R¹⁷, —H, and —COOR¹⁵ wherein
R¹⁵ and R¹⁶ are independently selected from methyl and —H, and
R¹⁷ is selected from methyl, —H, 4-hydroxybutyl and 2-tetrahydrofuryl;
R⁹ is selected from phenyl which is optionally substituted, and hetaryl which is optionally substituted;
R¹⁰ is selected from alkoxy, —H, halogen, and alkyl, and
R¹¹ is selected from —H, hydroxyl, halogen, alkoxy and alkyl;
or R¹⁰ and R¹¹ are in ortho-position to each other and together form a —O(CH₂)ₙO— unit, with n=1 or 2,
or a derivative or a salt thereof.

18. The combined preparation claim 16, wherein said flavagline is a compound comprising a structure selected from the list consisting of formulas (II) to (IX):

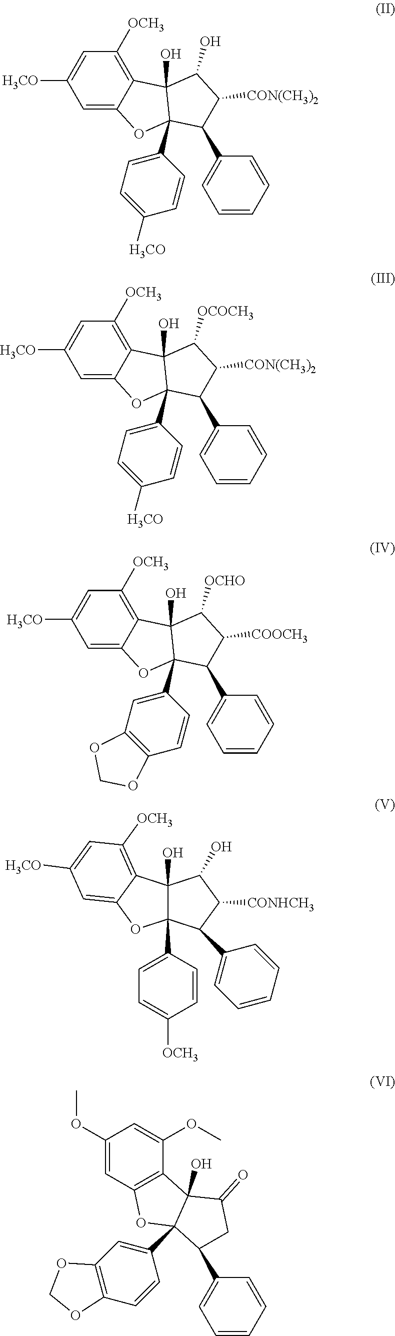

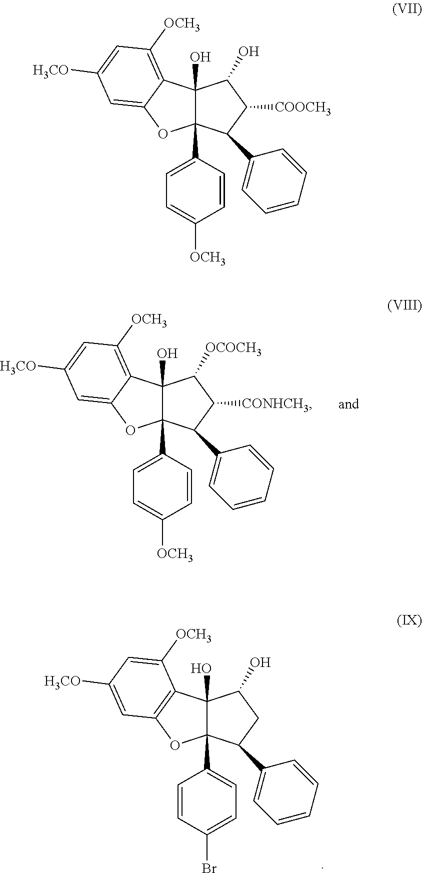

19. The combined preparation of claim 16, wherein said flavagline is (1R,2R,3S,3aR,8bS)-1,8b-dihydroxy-6,8-dimethoxy-3a-(4-methoxyphenyl)-N,N-dimethyl-3-phenyl-2,3-dihydro-1H-cyclopenta[b][1]benzofuran-2-carboxamide (Rocaglamide A) or a derivative thereof; or (1R,3S,3aR,8bS)-3a-(4-Bromophenyl)-6,8-dimethoxy-3-phenyl-2,3,3a,8b-tetrahydro-1H-cyclopenta[b]benzofuran-1,8b-diol (FL3) or a derivative thereof.

20. The combined preparation of claim 16, wherein said 2-deoxyglucose is 2-Deoxy-D-glucose ((4R,5S,6R)-6-(hydroxymethyl)oxane-2,4,5-triol).

21. The combined preparation of claim 16, wherein said combined preparation is a preparation comprising a mixture of said flavagline and said 2-deoxyglucose.

22. A method of treating cancer in a subject afflicted with cancer, comprising administering a flavagline and 2-deoxyglucose to said subject, thereby treating said cancer.

23. The method of claim 22, wherein said flavagline and said 2-deoxyglucose are in a combined preparation.

24. The method of claim 22, wherein said cancer is leukemia, lymphoma, HPV-related cancer, colorectal carcinoma, gastric cancer, pancreas cancer, lung cancer, brain cancer, or breast cancer.

25. The method of claim 22 wherein said 2-deoxyglucose is administered at a concentration of from 0.01 mM to 20 mM.

26. A process for the preparation of a combined preparation according to claim 16, comprising the step of mixing a flavagline and 2-deoxyglucose.

27. The process of claim 26, further comprising the step of formulating the mixture of a flavagline and 2-deoxyglucose as a pharmaceutical composition.
